(19) United States
(12) Patent Application Publication
(10) Pub. No.: US 2026/0346853 A1
(43) Pub. Date: Nov. 5, 2026

(54) PHYSIOLOGICAL MONITORING AND RESPONSE PHASE SEGMENTATION SYSTEMS

(71) Applicant: Toyota Motor Engineering & Manufacturing North America, Inc., Plano, TX (US)

(72) Inventors: Hossein Hamidi Shishavan, Ann Arbor, MI (US); Frederico Marcolino Quintao Severgnini, Ann Arbor, MI (US); Paul D. Schmalenberg, Ann Arbor, MI (US); Ercan M. Dede, Ann Arbor, MI (US)

(73) Assignee: Toyota Motor Engineering & Manufacturing North America, Inc., Plano, TX (US)

(21) Appl. No.: 19/195,596

(22) Filed: Apr. 30, 2025

(51) Int. Cl. B60W 40/08; A61B 5/00; A61B 5/0205; A61B 5/16; A61B 5/18; B60W 50/14; B60W 50/16; G16H 40/67; G16H 50/30

(52) U.S. Cl. CPC B60W 40/08; A61B 5/0002; A61B 5/0205; A61B 5/165; A61B 5/18; A61B 5/6893; A61B 5/7207; A61B 5/7282; A61B 5/746; B60W 50/16; G16H 40/67; G16H 50/30; B60W 2040/0872; B60W 2050/143; B60W 2050/146; B60W 2540/221; B60W 2556/45

(57) ABSTRACT

In one embodiment, obtaining physiological signal data; preprocessing the physiological signal data to remove motion artifacts; extracting, via signal processing, one or more biomarker data from the physiological signal data; and phase segmenting a first biomarker data of the one or more biomarker data into general adaptation syndrome (GAS) phases.

### BACKGROUND

### Field

Accurate monitoring and analysis of human physiological signal data (physiological data) is performed in a variety of industries, including healthcare, vehicular hazard reductions and operations, security and defense, education and training, sports and performance, and employment health, along with a myriad of others. However, modern systems used in these industries, including those deploying machine learning (ML) models and artificial intelligence (AI), are limited in their ability to accurately assess human conditions due to issues such as lack of transparency and the sheer volume of data required.

ML and AI are deployed by the industries described above to aid or improve outcomes. ML models are at times used to monitor and assess human physiological states. To do this ML models can be trained using various techniques, many of which rely on labeled data, e.g., in supervised training, where inputs are mapped to a correct or labeled output. When training using labeled data, an ML model repeatedly adjusts its internal parameters to minimize discrepancy between the generated predictions and the expected outputs (that are based on the labels) to improve its performance.

Current systems and models deployed in industry fail to accurately determine human conditions as a whole and beyond the immediate physiological data collected. Accordingly, models and systems for improved physiological data analysis to ascertain emotional states may be desired for a variety of industries.

### BRIEF SUMMARY

In one embodiment, a method includes obtaining physiological signal data; preprocessing the physiological signal data to remove motion artifacts; extracting, via signal processing, one or more biomarker data from the physiological signal data; and phase segmenting one or more biomarker data into general adaptation syndrome (GAS) phases.

In another embodiment, a processing system includes a memory including computer-executable instructions and a processor configured to execute the computer-executable instructions and cause the processing system to obtain physiological signal data, preprocess the physiological signal data to remove motion artifacts, extract, via signal processing, one or more biomarker data from the physiological signal data, phase segment a first biomarker data of the one or more biomarker data into general adaptation syndrome (GAS) phases, generate one or more respective similarity scores for each GAS phase of the one or more GAS phases based on at least one of a similarity to other GAS phases of the GAS phases or on a similarity to baseline data, and label the first biomarker data based on the GAS phase to generate first labeled data.

In another embodiment, a vehicle includes a vehicle body, one or more sensors, and a processing system coupled with the one or more sensors. The processing system includes a processor configured to execute computer-executable instructions and cause the processing system to obtain physiological signal data of an occupant of the vehicle from the one or more sensors, determine an emotional state of the occupant based on phase segmented biomarker data of the physiological signal data, and enforce a response mechanism comprising one or more of an output of an audio-visual alert, an output of a haptic alert, change in vehicular control, or an adjustment of vehicle movement based on the emotional state.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a simplified graph depicting GAS.

FIG. 2 illustrates an example phase segmented graph of a biomarker, according to one or more embodiments described and illustrated herein.

FIG. 3 illustrates example phase segmented graphs for two different biomarkers, according to one or more embodiments described and illustrated herein.

FIG. 4 illustrates a graph of the types of changes associated with types and intensity of different emotional states as detected by phase segmentation, according to one or more embodiments described and illustrated herein.

FIG. 5 illustrates a graph of a phase segmented physiological response across time, according to one or more embodiments described and illustrated herein.

FIG. 6 illustrates deploying the affective response tools as part of a health care system, according to one or more embodiments described and illustrated herein.

FIG. 7 illustrates deployment of the affective response tools in a vehicle system, according to one or more embodiments described and illustrated herein.

FIG. 8 illustrates a method of emotional state monitoring and response based on phase segmentation, according to one or more embodiments described and illustrated herein.

FIG. 9 illustrates a method of emotional state monitoring and response for an occupant of a vehicle based on phase segmentation, according to one or more embodiments described and illustrated herein.

FIG. 10 depicts an example processing system configured to perform various methods described herein, according to one or more embodiments described and illustrated herein.

### DETAILED DESCRIPTION

General adaptation syndrome (GAS) describes the body's physiological adaptation and response to stressors, e.g., stimuli or events, through three distinct phases to restore homeostasis. The three phases may include, alarm, resistance, and exhaustion that are preceded and proceeded by normal phases. Each phase possesses unique characteristics that can provide insights into the intensity and types of emotions experienced by an individual. Analyzing these various phases of GAS, can therefore be used to derive physiological and emotional data and assess physiological and emotional states. However, GAS is not utilized in physiological and emotional data monitoring.

Emotional state monitoring is desired and useful in a range of industries and systems, including public health and healthcare, vehicular operations, security and defense, education and training, sports and performance, and employment health, along with a myriad of others. However, current systems and models fail to accurately monitor and assess physiological and emotional states of individuals, including general wellbeing and health responses to stressors. Systems and methods for emotional state monitoring and responses (affective response tools) are disclosed herein that use a GAS model to derive emotional state data from physiological state data to monitor and respond to emotional states.

In the realm of ML, many types of ML training processes rely on accurately labeled training data. However, accurately labeled training data are difficult to obtain for specialized domains such as emotional monitoring. This may be due to examiner biases and personal and cultural norms and perceptions. The affective response tools provide accurately labeled training data to train ML models in the domain of human emotions. Data analysis and phase segmentation based on GAS phases are used to provide more accurate and comprehensive training data labels. Phase segmentation or phase segmenting refers to a process of segmenting biomarker data into various GAS phases. ML models trained on more accurate data generate superior results in monitoring and responding to human emotional states.

Emotional states are derived from physiological data by analyzing the physiological data according to the GAS model. This occurs by deriving the various phases of the GAS model using phase segmentation. The characteristics of the various phases are then analyzed and interpreted to determine emotional state of an individual at each phase, and at points prior to and subsequent to the analyzed phases. Additionally, based on this, labels may be added to the analyzed physiological data associating the physiological data or portions thereof to the determined emotional states.

Physiological patterns can behave differently across individuals, meaning there is no singular, fixed pattern or behavior for every event-person pairing. Emotional and physiological responses can shift based on context, day, and countless other variables. To address this, Emotional states are assigned labels based on phase-specific biomarker behaviors, allowing the derivation of emotional states to be based beyond the assumption that a stimulus always triggers the same affective response. Instead, emotional states are inferred dynamically by emotional states being mapped based on biomarker trajectories across different phases, rather than relying on static snapshots. Assigned labels thus are able to reflect individualized physiological patterns, capturing personal variations in emotional responses.

Over time, a library of physiological responses can be built, enabling more accurate matching between types of stressors and the corresponding response patterns. The specific emotional label—whether called ‘anxiety,’ ‘fear,’ or otherwise—becomes less important; what matters is the similarity in the physiological state. For example, the body's response to an examination and to a job interview might be nearly identical, even if the emotional labels differ.

Furthermore, the affective response tools herein can leverage already collected physiological data, e.g., in healthcare settings, and based on GAS models to generate accurate patient monitoring of emotional states. This produces improved response times and more effective interventions to better patient outcomes.

In vehicles, accurate assessment of occupants'emotional states can be vital to ensure the proper operation of the vehicle. However, vehicular systems generally lack accurate and comprehensive monitoring solutions of emotional states. Embodiments of the present disclosure provide a vehicle having sensors. A processing system or control unit of the vehicle obtains physiological signal data of an occupant from the sensors, and determines an emotional state of the occupant based on GAS phases applied to the collected sensor data. The processing system of the vehicle may then effectuate or enforce a response mechanism that can include a number of actions such as an output of an audio-visual alert, an output of a haptic alert, a change in vehicular control or movement based on the emotional state(s) identified, and initiation of or increase in autonomous vehicle control and management.

Various embodiments of systems, methods and vehicle employing GAS are described in detail below.

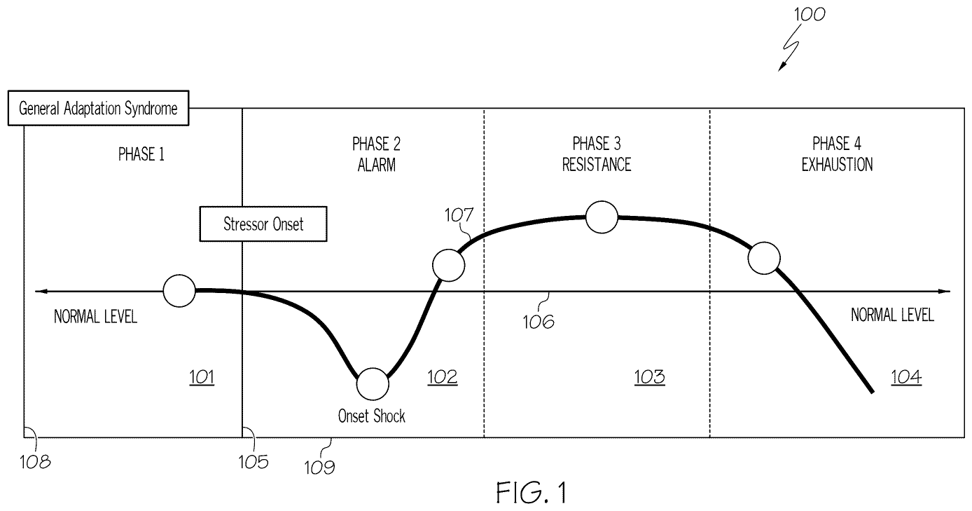

FIG. 1 illustrates a simplified graph depicting GAS. The graph 100 depicts various phases 101-104 of GAS. GAS is generally outlined as a three-stage model that describes the body's adaptation to various stressors. Depicted here are the three phases, an alarm phase 102 (sometimes referred to as a compensation phase), a resistance phase 103 and an exhaustion phase 104 (sometimes referred to as a decompensation or recovery stage). Preceding the phases 102-104 is the normal phase 101. The normal phase 101 is the body's normative immune resting state prior to a stressor onset 105. The normal phase 101 itself may be a result of previous adaptation(s) to stimuli. The Y-axis 108 describes the intensity of the body's stress (fight or flight) response, and the X-axis 109 represents time. The GAS model is one example of a model that depicts an individual's response to a stressor. Many other models may be implemented that are not described herein.

Once a person experiences a stressor, which can include an event or stimuli, e.g., an internal event (mental or psychological) or an external event (originating outside of the human body), at the stressor onset 105, the body moves from its normal phase 101 to the alarm phase 102. During the normal phase 101, the body's current stress resistance 107 is at a baseline stress resistance level 106. After stressor onset 105, the body enters the alarm phase 102, where the body is initially in shock and the body's current stress resistance 107 falls under the baseline stress resistance level 106. Still within the alarm phase 102, the body then enters a fight or flight response where the body's current stress resistance 107 increases above the baseline stress resistance level 106 and the body releases stress hormones including adrenaline and cortisol to prepare itself to confront or flee the stressor.

The body then enters the resistance phase 103, where the body attempts to adapt to the stressor, and the body's current stress resistance 107 increases to a level that is higher than the baseline stress resistance level 106. Because the body is adapting to the stressor, the body continues to produce a high level of stress hormones and expends considerable energy and resources and the body's current stress resistance 107 remains in an elevated state. In the exhaustion phase 104 after the resistance phase 103, the body reaches a point where its resources have been depleted due to exposure to the stressor and attempting to adapt to the stressor, the body's current stress resistance 107 of the body therefore drops to below the baseline stress resistance level 106. In cases where the stressor is either removed promptly or does not exceed the individual's physiological or psychological coping capacity, the stress response does not necessarily progress into an exhaustion phase. Instead, the individual transitions directly into a recovery phase.

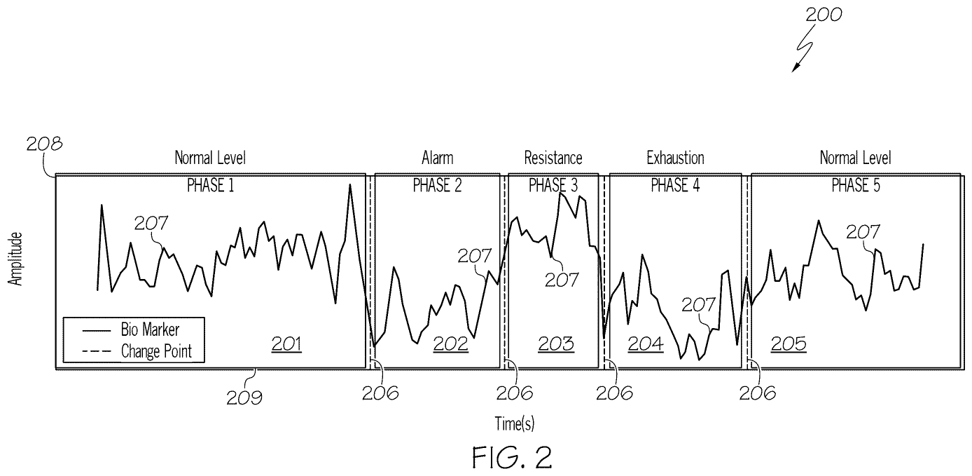

FIG. 2 illustrates an example phase segmented graph 200 of a biomarker, according to one or more embodiments described and illustrated herein. The example phase segmented graph 200 and its associated data are generated by the affective response tools described herein with phase segmentation. A biomarker is a measurable indicator of a physiological condition, reaction, or process. For example, blood pressure, heart rate and skin conductivity are biomarkers.

The GAS phases depicted in FIG. 2 include phases 201-205 of a biomarker. Five phases are depicted including a normal phase 201 preceding the three phases of GAS, an alarm phase 202, a resistance phase 203, and an exhaustion phase 204. The phases 201-204 may correspond with the normal phase 101, the alarm phase 102, the resistance phase 103, and the exhaustion phase 104 of FIG. 1, respectively. The normal phase 205 is another normal phase subsequent to the exhaustion phase 204 and depicts the body's response as it enters back into a normal level of resistance. The affective response tools herein may separate or define these phases 201-205 with marker(s) 206. The marker(s) 206 may include data labels, tags, identifiers, annotations, flags, tokens, metadata, annotations, and the like to identify or categorize each of the phases 201-205. The Y-axis 208 of the graph represents an amplitude of the biomarker 207 and the X-axis 209 represents time. The marker(s) 206 may depict critical transition where the underlying physiological data reflects a shift from one emotional or regulatory state to another.

The amplitude of the biomarker 207 changes across the various phases 201-205. These changes may be analyzed by the affective response tools herein to not only determine phase(s) of the biomarker 207 but also additional information of the biomarker 207 in the analyzed phase(s) well. Accurate labeling of such data can then be performed, e.g., via the marker(s) 206, to add to a labeled data set to be used to train an ML model. The biomarker phases 201-205 can be identified via phase segmentation as performed by the affective response tools.

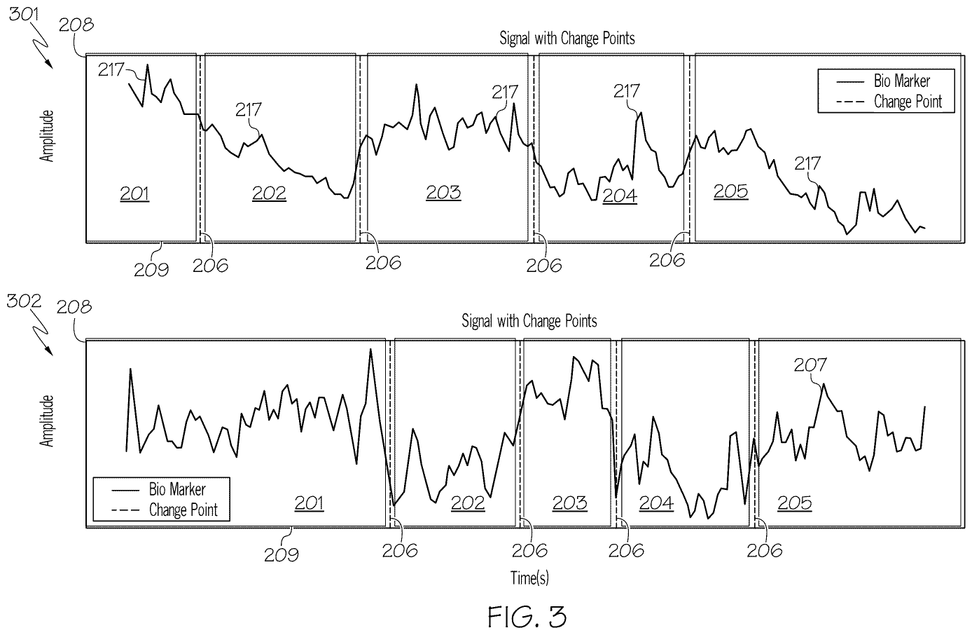

FIG. 3 illustrates example graphs 301 and 302 depicting GAS phases for two biomarkers 217 and 207, respectively as derived by phase segmentation, according to one or more embodiments described and illustrated herein. The example graph 302 corresponds to the example phase segmented graph 200 and is referred to interchangeably herein.

The GAS phases of each biomarker 207 and 217 can differ in their amplitudes (Y-axis 208) and can differ in their phase timings across the same total period of time of 175 (X-axis 209). While a stressor occurs at a specific time point, physiological responses across different biomarkers can manifest with varying temporal delays. For example, electrodermal activity (EDA) may react almost instantaneously to a stressor, reflecting immediate sympathetic nervous system arousal, while cortisol levels may peak only minutes after the onset of the stressor due to the slower dynamics of the hypothalamic-pituitary-adrenal (HPA) axis. Differences between different biomarkers also include variations in onset, duration, and distribution of each of the phases among the biomarkers 207, 217. For example, after experiencing high arousal emotions, individuals may take longer to return to baseline emotional states, e.g., the biomarker 207 exits the exhaustion phase 204 to enter the normal phase 205 at around 135-140 seconds, while the biomarker 217 exit the exhaustion phase 204 to enter the normal phase 205 at around 125 seconds.

Other information can be gleaned from the GAS phases and the variations between the biomarkers 207, 217. For example, in regards to the duration of the resistance phase 203, the shorter resistance phase 203 of the biomarker 207 indicates that a coping mechanism was effective in resolving the stress quickly. Conversely, a longer resistance phase 203, e.g., the resistance phase 203 of the biomarker 217, indicates that the body had an extended activation of the body's stress-response systems (such as elevated cortisol levels, increased heart rate, increased blood pressure) indicating that the individual is emotionally more stressed. A longer resistance phase 203 generally depletes the body's resources and can lead to negative health outcomes such as burnout, or weakened immunity, information vital to health management.

The biomarkers 207, 217 are phase segmented and can provide information on how different organs in the body react to stress and adapt over time based on the timings and amplitude of each of the phases assessed.

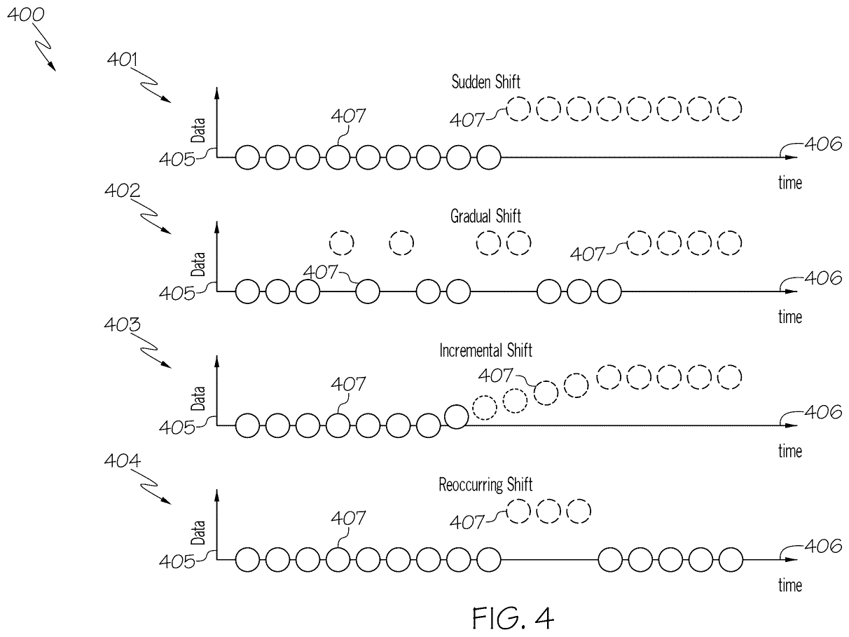

FIG. 4 illustrates a collection of graphs 400 depicting types of changes as responses to a stressor possible in sample data. These changes depicted in the graphs 400 are based on detected and isolated changes in the sample data, according to one or more embodiments described and illustrated herein. The graphs 401-404 are derived from the phase segmented data as described in the example graphs 301 and 302 of FIG. 3. The graphs 401-404 are depicted with a Y-axis 405 that represents a value of a biomarker, and the X-axis 406 that represents time. Each of the graphs 401-404 an example of how underlying data may change from one phase to another, e.g., one GAS phase to another, e.g., patterns of changes in data from one phase to another. The affective response tools described herein can use these patterns of change to determine whether a biomarker has changed phases e.g., from one GAS phase to another. The affective response tools described herein, including phase segmentation, can therefore identify different types of changes, e.g., the changes shown in graphs 401-404.

Importantly, the nature of the changes themselves detected or identified (whether sudden or slow, etc.) can carry meaningful information about an individual's physiological and mental states, as well as stressor characteristics. For example, a sudden shift can indicate acute reactions to an immediate, high-intensity stressor (e.g., threat, fear), while a gradual change can reflect more complex or prolonged stress exposures (e.g., sustained workload, social pressure). Therefore, both when a transition or change between phases occurs, and also how it occurs provides information for accurate emotional state labeling and for characterizing the type of stressor influencing a system/individual.

Graph 401 depicts a pattern of a coping mechanism with a sudden shift in the biomarker 407, the coping mechanism are based on readings of the biomarker 407 that are generally at a consistently low amplitude before suddenly being detected at a consistently higher amplitude within a specific phase. Graph 402 represents a gradual shift pattern in a coping mechanism, with the readings of the biomarker 407 being initially consistently low with a small but gradually increasing number of higher amplitude readings that gradually increase in frequency interspersed among the low amplitude readings until most of the readings are at a consistently high amplitude within a specific phase.

Graph 403 depicts a pattern of a coping mechanism with an incremental shift in the biomarker 407, where the readings of the biomarker 407 are initially at a low amplitude but incrementally and continually increase to reach a consistent higher amplitude within a specific phase. Graph 404 depicts a pattern of a coping mechanism of a recurring shift in the biomarker 407, where the readings of the biomarker 407 initially at a consistent low amplitude but then suddenly shift to a consistent higher amplitude but then shift again to a consistent low amplitude within a specific phase.

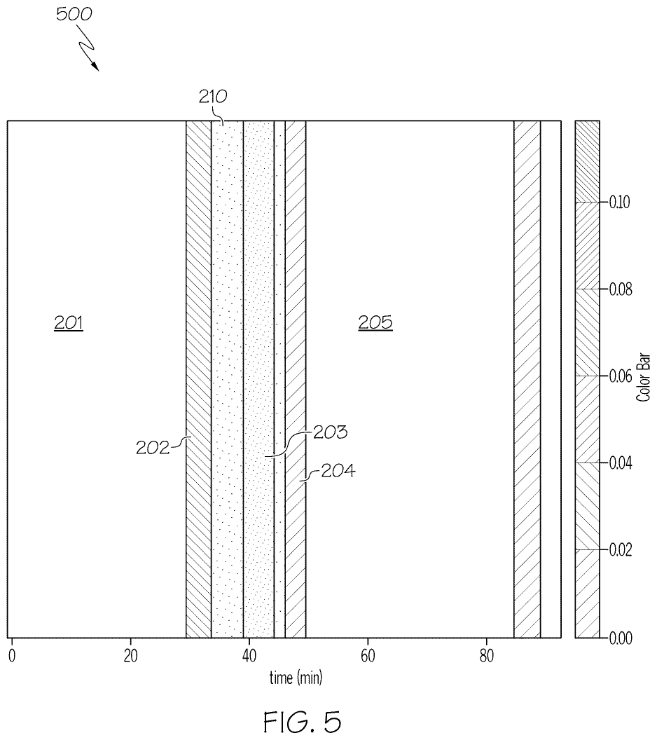

FIG. 5 illustrates a time graph 500 of a phase segmented physiological response over time, according to one or more embodiments described and illustrated herein. FIG. 5 depicts results of phase segmentation of one or more biomarker data of physiological signal data.

The time graph 500 has an X-axis 501 that represents time where the relative time length of each phase of the GAS phases can be compared. Once temporally aligned, each phase identified by phase segmentation can be scored, and these scores can either be averaged across all biomarkers as illustrated in FIG. 5.

The phases shown correspond with the phases 201-205 of FIGS. 2-3. In the example of the time graph 500, the biomarker begins at the normal phase 201, then moves to the alarm phase 202, and then there is an onset of a stressor at bar 210, which is followed by a bar representing the resistance phase 203 and then another bar representing the exhaustion phase 204, followed by a return to the normal phase 205. The bar 210 indicates when the actual stressor occurred. However, in this instance an individual consciously or subconsciously anticipated the event before it happened. As a result, the individual's reaction begins earlier than the onset of the stressor.

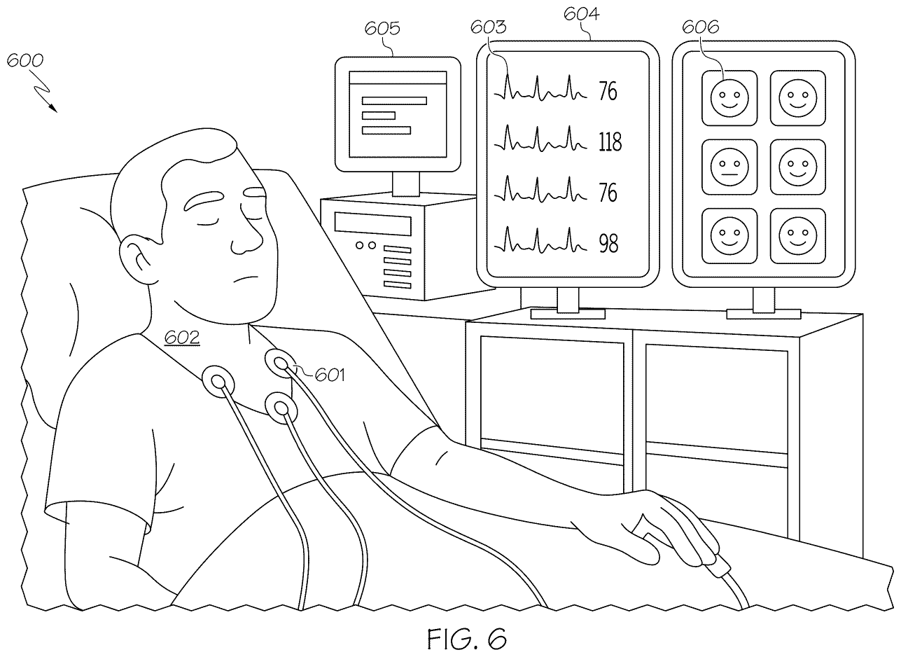

FIG. 6 illustrates deployment of the affective response tools as part of a health care system 600, according to one or more embodiments described and illustrated herein. The health care system 600 can include sensor(s) 601 placed on a patient 602. The physiological data 603 of the patient 602 can be output on a display 604. The physiological data 603 can also be sent to a processing system 605, e.g., affective response tool processing system, that can derive emotional states by analysis of physiological data 603 that is phase segmented. The health care system 600 can also send the derived emotion data to an regulatory data display 606 to display in real-time the emotional states of the patient 602 as derived by the processing system 605. For example, it may display a number of icons representing stress, mood, and overall well being on the regulatory data display 606.

For example, the processing system 605 may receive the physiological data 603 from the sensor(s) 601 and implement the affective response tools to phase segment the physiological data 603 into various phases, and analyze the phases to determine the emotional state of the patient 602, and then cause the emotional state to be displayed on the regulatory data display 606. In some embodiments, other response mechanisms may be executed based on the emotional or physiological state of the patient 602. For example, an audio-visual alert may be output to various staff, bed or seating position of the patient 602 may be adjusted automatically, a health care plan for the patient 602 may be adjusted or modified, drug dosages being dispensed or provided to the patient 602 may be adjusted.

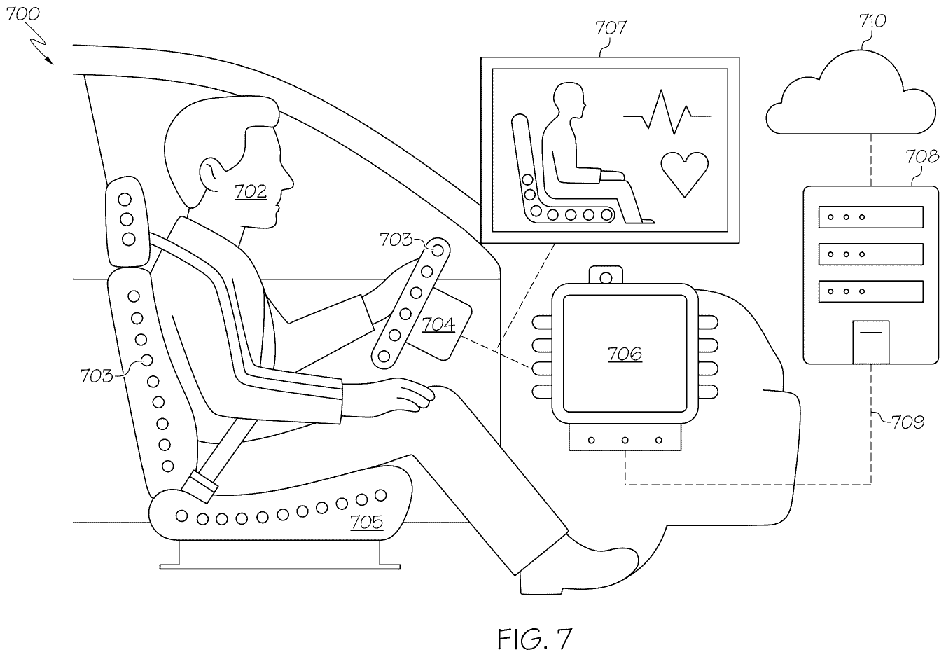

FIG. 7 illustrates deployment of the affective response tools in a vehicle system 700, according to one or more embodiments described and illustrated herein. The vehicle system 700 includes a vehicle 701 that may include an occupant 702, e.g., a driver or a passenger. The occupant 702 can have their physiological signal data collected by sensor(s) 703, e.g., sensors on a steering wheel 704 that the driver is in contact with. In some embodiments, the sensor(s) 703 may be integrated with a seat 705 or any other part of the vehicle 701 or the sensor(s) 703 can be contactless.

The sensor(s) 703 may send their data to a processing system 706, e.g., an electronic control unit (ECU) or other processing unit, that receives the physiological sensor data and utilize affective response tools and can display this data on a display 707, e.g., dashboard screen, or sends the physiological sensor data to an external server system 708, e.g., via a network interface 709. The external server system 708 may be part of a cloud system 710. The external server system 708 may utilize affective response tools as described herein and send the results back to the vehicle 701, via the network interface 709. The network interface 709 connects the external server system 708 to the processing system 706, which then causes a display or otherwise audio-visually output of the emotional phase segmented data obtained by the affective response tools described herein.

The vehicle system 700 can also generate automatic vehicle control signals. For example, the vehicle system 700, can trigger advanced driver-assistance systems (ADAS) in response to affective response tools determining presence or elevation of an emotional state, e.g., stress or fatigue. For example, the ADAS can receive instructions to take over control of the vehicle and its movement, or stop/park it at the nearest stop space. In some embodiments, an autonomous driving system could adjust its decisions based on the individual's physiological or emotional state.

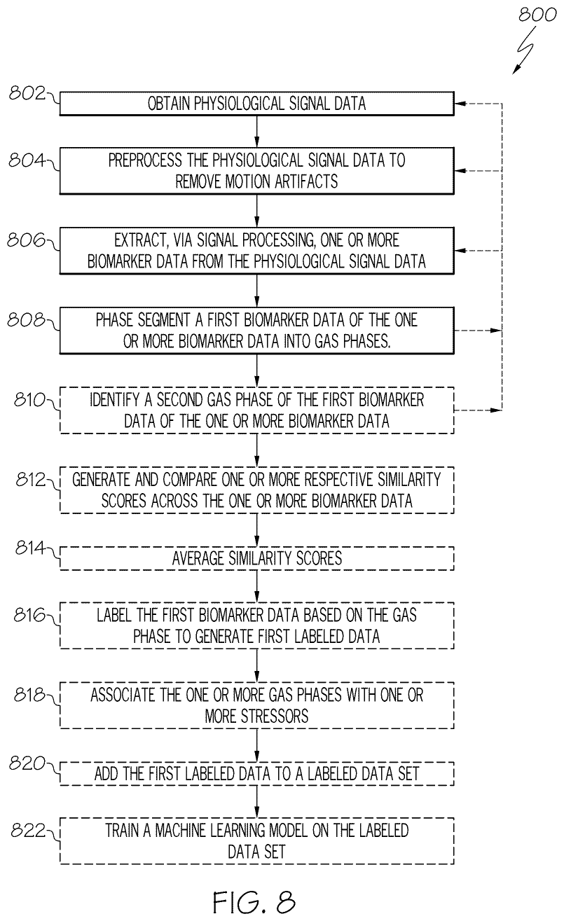

FIG. 8 illustrates a method 800 of determining emotional states with the affective response tools based on phase segmentation of a biomarker, according to one or more embodiments described and illustrated herein. The method 800 may include or correspond to the affective response tools disclosed herein and can be performed by a processing system or the systems 600-700 of FIGS. 6 and 7. It is to be understood that blocks 802-822 are not listed in any particular order, and that they can be performed in any order. It is to be understood that blocks or arrows represented with dashed lines represent optional embodiments that may or may not be included in the method 800.

The method 800 begins at block 802 with obtaining physiological data, e.g., physiological signal data of a person. Obtaining physiological data can include receiving data from one or more sensors or probes, e.g., the sensor(s) 601 of FIG. 6 or 703 of FIG. 7. Sensor(s) may include one or more sensors of various types to collect different physiological data including cardiovascular sensors, e.g., electrocardiogram sensors (ECG), pulse oximeters (SpO₂), heart rate (HR) sensors, and blood pressure (BP) sensors; neurological sensors, e.g., electroencephalograms (EEG) electrode or sensors, electromyography (EMG) muscle tissue sensors, electrooculography (EOG) sensors; respiratory sensors, e.g., respiratory rate (RR) sensors, end-tidal CO₂ (ETCO₂) sensors, chest expansion sensors; thermal and metabolic sensors, e.g., body temperature or skin temperature sensors, infrared thermography sensors; electrodermal sensors, e.g., galvanic skin response (GSR) sensors, hydration sensors; glucose and biochemical sensors, e.g., blood glucose sensors, lactate sensors, electrolyte sensors; motion sensors, e.g., accelerometer, gyroscope, magnetometer, force-sensitive resistor, and strain gauge sensors. The sensor(s) can transmit or send the readings of the physiological data to a processing system or other device which obtains the physiological data.

After receiving the physiological data, the method 800 initiates the affective response tools by preprocessing the received physiological data to remove motion artifacts, e.g., distortions or noise in the data. Preprocessing the physiological signal data can include using filtering techniques such as low-pass/high-pass filters, adaptive filters, and wavelet transforms. A low-pass filter allows lower frequency signals to remain while filtering higher frequency signals, meanwhile a high-pass filter allows higher frequency signals to remain while filtering lower frequency signals. Adaptive filters are filters that dynamically adjust its parameters based on errors it encounters in the data to minimize cost functions and generate optimal outputs. For example, adaptive filters rely on adaptation algorithms or an iterative process to update internal parameters. Wavelet transforms decompose a signal into smaller signals to analyze each signal in detail and then handling each signal based on this detailed analysis.

The method 800 then continues to block 806 to extract, via signal processing, one or more biomarker data from the physiological data that was preprocessed at block 804. Blocks 804 and 806 can be performed by ML models as well as the processes described above. For example, neural networks can be trained to remove noise or to extract relevant biomarkers from data. The signal processing that is deployed at block 806 can include Fourier transform, power spectral density (PSD) analysis, and time-domain statistical measures. Fourier transform analyzes the frequency components of signals. PSD analysis quantifies the distribution of power across frequency components of the preprocessed physiological data. Time-domain statistical measures include applying one or more of mean, standard deviation, root mean square (RMS), and entropy, to the physiological data that was preprocessed at block 804.

Once the one or more biomarker data are extracted from the physiological data, the method 800 continues to block 808 to phase segment a first biomarker data of the one or more biomarker data into the GAS phases, e.g., to output data that can be graphed as example phase segmented graph 200 of FIG. 2. It is to be understood that the first biomarker data, the second biomarker data, and so forth can refer to data associated with one biomarker or multiple biomarkers, can refer to a dataset of biomarker signals or readings, or can refer to one individual reading (e.g., sensor reading).

Phase segmenting of the first biomarker data can include applying the GAS concept via a non-parametric technique, such as a kernel-based segmentation technique. The kernel-based technique is flexible and can be applied to a wide range of sensors being used and can adapt to data with complex patterns and variations without having to rely on predefined parameters. Phase segmentation at block 808 can include using the kernel-based technique to identify each of the phases of the GAS phases and mark each phase with a tag, label or other data marker, e.g., the marker(s) 206 of FIG. 2.

The kernel-based segmentation technique may be similar to kernel-based segmentation techniques applied in other fields, and it shares similarities with existing applications. However, it is to be understood that phase segmentation as described herein is not restricted to kernel-based segmentation alone. In general, segmentation methods can be categorized under two broad classes: parametric and nonparametric techniques. Generally, the kernel-based segmentation technique is a nonparametric technique, which can be advantageous for physiological data analysis as it does not require any assumptions about the underlying data distribution allowing it to be flexible in implementation. This flexibility is advantageous given that biomarker-signals often exhibit complex, non-Gaussian, and nonstationary behavior that would be poorly captured by rigid parametric techniques. Nevertheless, depending on the research goals and the data characteristics, alternative segmentation techniques (whether parametric or nonparametric) can be integrated or used by the affective response tools described herein.

In some embodiments, the phase segmentation includes identifying a first GAS phase of a first biomarker data of the one or more biomarker data, e.g., one of the phases 201-205 of FIG. 2, and can also include marking the first GAS phase of the first biomarker data based on at least one of a start or an end of the first GAS phase. Phase segmentation at block 808 can also include identifying multiple phases, e.g., a second GAS phase such as one of the phases 201-205, a third, a fourth, and a fifth GAS phase of the GAS phases depending on the embodiment. Phase segmentation can also include marking the identified phases. Markers can be set to identify a start or an end of an identified phase.

In optional embodiments, at block 810, phase segmentation can be performed on multiple bio markers, e.g., a second biomarker, to produce phase segmentation of multiple bio markers, e.g., as depicted in FIG. 3 with the example graphs 301 and 302. Optionally, after phase segmentation of a biomarker, e.g., at block 808 or at block 810, then the blocks 802-806 can be repeated and additional biomarkers, e.g., from additional physiological data, can be phase segmented into GAS phases.

In optional embodiments, at block 812, the method 800 can include generating one or more respective similarity scores for each GAS phase of the GAS phases, the similarity score can be based on at least one of a similarity to other GAS phases or a similarity to baseline data, e.g., comparison to a normal resistance level or other available reference or historical data. For example, similarity scores of the phases are compared with each other and against any baseline data beyond the region of interest. Similarity scores can provide insights into patterns of emotional adaptation, revealing how the body's physiological responses vary in intensity and direction over time. The similarity scores can be compared using the Kullback-Leibler divergence, which quantifies by how much one probability distribution differs from a reference distribution, or using Dynamic Time Warping (DTW), which measures differences between time series. Similarity measures can be applied not only within the immediate temporal window but also between current data and historical records (ranging from days to even years prior). When applied at finer temporal resolutions, similarity analysis can capture short-term dynamics in emotional states, such as changes in arousal or valence levels. For example, a shift toward persistently higher arousal could indicate escalating stress levels that may pose a risk to well-being, whereas movement toward more regulated or positive states may reflect improvements in emotional resilience and overall health. Thus, similarity-based approaches offer a versatile tool for both macro-level (longitudinal health monitoring) and micro-level (moment-to-moment emotional regulation) analyses. It should be noted that other analytical techniques, such as correlation-based methods and other similarity measures, can also be employed for this section.

Results based on phase segmenting the one or more biomarker data of the physiological signal data is depicted in FIG. 5. Once the one or more biomarker data is phase segmented based on detected changes, e.g., the change(s) as depicted in graphs 400 of FIG. 4, not all segments are necessarily related to a response to the stressor. Some identified phases segments may simply reflect normal physiological variations, such as natural body metabolism, unrelated to the stressor. Therefore, a similarity score is calculated, e.g., at block 812, between phases to assess whether a given phase matches an expected stressor response pattern closely enough, e.g., within a margin of error.

Similarity scoring can also be applied across phases of a physiological response to identify periods most strongly associated with stressor response. For instance, the resistance phase may show the highest dissimilarity from baseline due to higher arousal compared to preceding or subsequent phases. After calculating similarity scores for each phase, another step may include alignment across different biomarkers. Because various physiological signals have inherent time delays relative to each other (e.g., skin conductance changes may precede heart rate changes), it is necessary to temporally align detected onsets of the identified phases to ensure proper comparison.

Once temporally aligned, each phase identified by phase segmentation can be scored, and these scores can either be averaged across all biomarkers (as illustrated in FIG. 5) or further processed using ML algorithms. ML approaches can help identify which biomarkers have the strongest relationship to the stressor, allowing for adaptive weighting. For example, heart rate might contribute more significantly to stress responses than skin temperature, and the model could assign higher weights to heart rate accordingly.

Alternatively, manual or data-driven weighting strategies can be applied to enhance the interpretability and sensitivity of the analysis, ensuring that each biomarker's contribution accurately reflects its physiological relevance to the emotional or stress-related event.

In optional embodiments, the similarity scores are averaged, e.g., after temporal alignment as described above, at block 814 across different biomarkers to determine the body's overall response to a stressor, including an emotional state of an individual based on the average scores, For example, an average can be taken across different biomarkers based on specific phases identified of each biomarker or averages based on time in association with a stressor.

In optional embodiments, at block 816, the one or more biomarkers that had phase segmentation performed on them can be labeled, e.g., marker(s) 206 can be added to each phase that is identified. For example, labels can be added to each phase of the biomarker, to the biomarker, or to multiple biomarkers or phases. It should be noted that that some or none of the phases may be detected. For example, based on the physiological signal data only one phase is detected, or none at all. In complex situations, more than three phases may be identified, appearing across multiple consecutive segments.

In optional embodiments, at block 818 the phases identified by phase segmentation can be associated with one or more stressors. This can be done by receiving a user input, e.g., a response to an information request, e.g., a push notification, sent by the affective response tools. The user or individual can confirm or provide information on the stressor and their feelings/reactions in the response which can be used by the affective response tools herein to associate specific biomarker timings or data to information provided by the user or individual. Alternatively, the associating can be based on data collected from a sensor, e.g., Heart rate goes up immediately after a car crash and therefore the sensor reading is associated with the stressor event. The associations to stressors may also be labeled with their association at block 816.

Once the data is labeled at 816, then at block 820 the labeled data can be added to a labeled data set, e.g., a data set mainly including labeled data used for training ML models.

One or more machine learning models can then, in optional embodiments, be trained at block 822 on the labeled data set, e.g., with supervised learning. The machine learning models trained can then be incorporated and deployed by or as part of various systems to derive emotional and physiological states of a person, e.g., the systems 600-700 of FIGS. 6-7.

In alternative embodiments, the method 800 can be utilized as a secondary method or used as a fine-tuning mechanism to determine emotional states, e.g., used to fine tune already trained algorithms or models (a primary method). First an emotional condition is identified using a primary method, e.g., changepoint detection, ML, etc. Then one or more of the blocks 802-822 are performed, e.g., to phase segment the biomarker data into phases at block 808, and then the results are compared to the already identified phases from the primary method. If the phases identified using phase segmentation match those from the primary method, the primary method results are affirmed, and parameters can be accordingly adjusted, otherwise the primary method has to attempt additional estimations to then be confirmed by the method 800 as described.

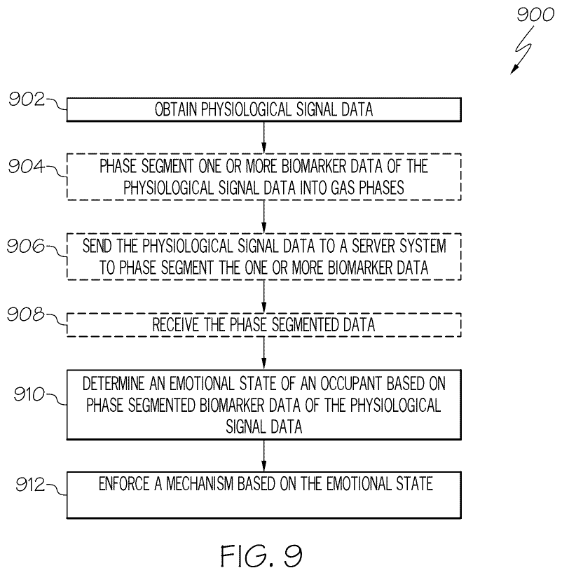

FIG. 9 illustrates a method 900 of determining emotional states of an occupant of a vehicle based on the affective response tools, according to one or more embodiments described and illustrated herein. The method 900 may include or correspond to the affective response tools disclosed herein and can be performed by a processing system or the vehicle system 700 of FIG. 7. It is to be understood that blocks 902-910 are not listed in any particular order, and that they can be performed in any order. The blocks 902-910 are represented with dashed lines are optional embodiments that may or may not be included in the method 900.

With reference to FIG. 9 in conjunction with FIG. 7, the method 900 begins at block 902 with obtaining physiological data, e.g., physiological signal data of an occupant of the vehicle such as a driver or passenger. Obtaining physiological data can include receiving data from the one or more sensors or probes, e.g., the sensor(s) 703. Block 902 may correspond with block 802 of method 800.

In optional embodiments, the method 900 proceeds to perform phase segmentation at block 904, e.g., by the processing system 706 of the vehicle 701, of one or more biomarker data of the physiological data obtained at 902 to derive one or more GAS phases of the one or more biomarker data. The phase segmentation may correspond to the phase segmentation of block 808 of method 800, details of which are not repeated for brevity.

In optional embodiments, the method 900 proceeds to block 906 and transmits or sends the physiological data to a server system, e.g., the external server system 708, for example via a network interface, e.g., the network interface 709, for the external server system 708 to perform phase segmentation of one or more biomarker data of the physiological signal data to derive one or more GAS phases of the one or more biomarker data.

In optional embodiments, the method 900 continues at block 908 with receiving the phase segmented one or more biomarker data, e.g., the vehicle 701 receives the phase segmented one or more biomarker data from the external server system 708, which in turn received the phase segmented data at block 906.

The method 900 then continues to block 910, with determining an emotional state of an occupant based on phase segmented one or more biomarker data, for example as discussed in relation to FIG. 4 based on analysis of changes in intensity associated with different types coping mechanisms as responses to a stressor, based on the similarity scores as described in relation to blocks 812-814 of FIG. 8, or based on associating the stressor to the response of phases identified e.g., as described in relation to block 818 of FIG. 8.

Finally, the method 900 continues to enforce an action, e.g., response mechanism, at block 910, if needed based on the determined emotional state at block 908. An action can include outputting an audio visual alert to the driver, e.g., informing the vehicle occupant of their emotional state, providing audio or displayed instructions to reduce hazards, e.g., provide instructions to relax or focus or, playing relaxation music, playing an alarm, adjust the lighting in the vehicle, adjusting the seat position, adjusting seat belts, or other parts of the vehicle. In some situations, the action may include completely or partially controlling or taking control of the vehicle, changing its movement, accelerating, decelerating, forcibly stopping the vehicle, or handing off driving functions to an AI agent or automated driving e.g., automated driving or AI that could factor in the driver's physiological state in the decisions it makes in control of the vehicle.

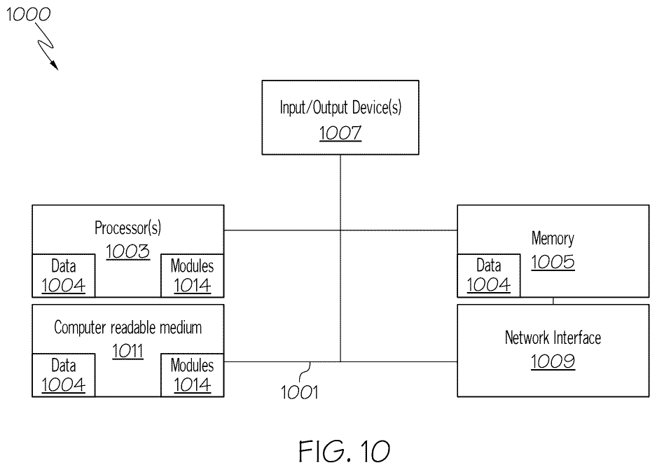

FIG. 10 depicts an example processing system 1000 configured to perform various methods described herein, including, for example, the methods 800-900 as described above with respect to FIGS. 8-9.

The processing system 1000 is generally an example of an electronic device configured to execute computer-executable instructions, such as those derived from compiled computer code, including without limitation personal computers, tablet computers, servers, smart phones, vehicles, smart vehicles, smart devices, wearable devices, augmented, virtual reality devices, and others. For example, the processing system 1000 may correspond to the processing system 706 of FIG. 7

In the depicted example, the processing system 1000 includes processor(s) 1003, memory unit(s) 1005, input/output device(s) 1007, e.g., display or audio device(s), network interface(s) 1009 through which the processor(s) 1003 is connected to network(s) (e.g., a local network, an intranet, the Internet, or any other group of processing systems communicatively connected to each other), and computer-readable medium 1011. In the depicted example, the aforementioned components are coupled by a bus 1001, which may generally be configured for data exchange amongst the components. The bus 1001 may be representative of multiple buses, while only one is depicted for simplicity.

The one or more processors 1003 are generally configured to retrieve and execute instructions, e.g., data 1004 stored in the memory units 1005, including local memories, computer-readable medium 1011, as well as remote memories and data stores. Similarly, the processor(s) 1003 are configured to store application data residing in local memories like the computer-readable medium 1012, as well as remote memories and data stores. The processor(s) 1003 may run module(s) 1014, e.g., an emotional state determination module, a phase segmentation module, a response mechanism enforcement module, etc. More generally, the bus 1001 is configured to transmit programming instructions and application data among the processor(s) 1003, input/output devices(s) 1007, network interface(s) 1009, and the computer-readable medium 1012. In certain embodiments, the processor(s) 1003 are representative of central processing unit(s) (CPU), graphics processing unit(s) (GPU), tensor processing unit(s) (TPU), accelerator(s), and other processing device(s).

Input/output device(s) 1007 may include any device, mechanism, system, interactive display, or various other hardware and software components for communicating information between processing system 1000 and a user of processing system 1000. For example, the input/output device(s) 1007 may include input hardware, such as a keyboard, display, touch screen, button, vehicle controls, microphone, speaker, or other device for receiving inputs from the user and sending outputs to the user.

The network interface(s) 1009 provide processing system 1000 with access to external networks and thereby to external processing systems. The network interface(s) 1009 can generally be any hardware or software capable of transmitting or receiving data via a wired or wireless network connection. Accordingly, the network interface(s) 1009 can include a communication transceiver for sending or receiving any wired or wireless communication.

### EXAMPLE CLAUSES

Implementation examples are described in the following numbered clauses:

Clause 1: A method, comprising obtaining physiological signal data; preprocessing the physiological signal data to remove motion artifacts; extracting, via signal processing, one or more biomarker data from the physiological signal data; and phase segmenting a first biomarker data of the one or more biomarker data into general adaptation syndrome (GAS) phases.

Clause 2. The method of clause 1, wherein the phase segmenting comprises identifying a first GAS phase of a first biomarker data of the one or more biomarker data; and marking the first GAS phase of the first biomarker data based on at least one of a start or an end of the first GAS phase.

Clause 3. The method of any of clauses 1-2, wherein the first GAS phase is one of a first normal level phase, an alarm phase, a resistance phase, an exhaustion phase and a second normal level phase.

Clause 4. The method of any of clauses 1-3, wherein the phase segmenting further comprises identifying a second GAS phase of the first biomarker data of the one or more biomarker data; and marking the second GAS phase of the first biomarker data based on at least one of a start or an end of the second GAS phase.

Clause 5. The method of any of clauses 1-4, further comprising generating one or more respective similarity scores for each GAS phase of the GAS phases based on at least one of a similarity to other GAS phases of the GAS phases or on a similarity to baseline data.

Clause 6. The method of any of clauses 1-5, wherein the first GAS phase is a resistance phase, wherein identifying the first GAS phase as the resistance phase is based on a similarity score of the resistance phase being lower than a threshold.

Clause 7. The method of any of clauses 1-6, further including comparing the one or more respective similarity scores to one another or to reference data based on a kullback-leibler divergence technique or a dynamic time warping technique.

Clause 8. The method of any of clauses 1-7, further comprising calculating a mean of one or more respective similarity scores for the first biomarker data.

Clause 9. The method of any of clauses 1-8, further comprising determining an overall physical response status to an event based on an overall mean of similarity scores of the one or more biomarker data.

Clause 10. The method of any of clauses 1-9, wherein the phase segmenting is based on at least one of a duration or a distribution of the first biomarker data.

Clause 11. The method of any of clauses 1-10, wherein the distribution comprises an amplitude of the physiological signal data.

Clause 12. The method of any of clauses 1-11, wherein the phase segmentation comprises a kernel-based technique applied to the one or more biomarker data.

Clause 13. The method of any of clauses 1-12, further comprising labeling the first biomarker data based on the GAS phase to generate first labeled data.

Clause 14. The method of any of clauses 1-13, further comprising adding the first labeled data to a labeled data set; and training a machine learning model on the labeled data set.

Clause 15. The method of any of clauses 1-14, further comprising associating the GAS phases with one or more events.

Clause 16. The method of any of clauses 1-15, further comprising phase segmenting a second biomarker data of the phase segmenting the first biomarker data into GAS phases.

Clause 17. The method of any of clauses 1-16, wherein the physiological signal data is obtained from one or more sensors.

Clause 18. The method of any of clauses 1-17, wherein the physiological signal data comprises one or more of electrocardiogram (ECG) data, Electrodermal (EDA) data, respiration data, skin temperature data, electroencephalogram (EEG) data, and blood glucose level data.

Clause 19. The method of any of clauses 1-18, wherein the preprocessing comprises a filtering technique.

Clause 20. The method of any of clauses 1-19, wherein the filtering technique comprises a low-pass filter, a high-pass filter, an adaptive filter, wavelet transforms, or machine-learning based methods.

Clause 21. The method of clauses 1-20, wherein the signal processing comprises at least one of Fourier Transform, Power Spectral Density Analysis, and Time-Domain Statistical Measures, or machine-learning based methods.

Clause 22. The method of any of clauses 1-21, wherein the one or more biomarker data comprises heart rate data from a diagnostic test, wherein the diagnostic test comprises one or more of an electrocardiogram (ECG), Electrodermal (EDA) activity, or an electroencephalogram (EEG).

Clause 23. The method of clauses 1-22, further comprising alerting one or more users based on an identified phase of the first biomarker data, wherein the identified phase is associated with a health status or an emotional status.

Clause 24. The method of clauses 1-23, further comprising output an audio-visual alert of health status information to one or more devices based on an identified phase of the first biomarker data.

Clause 25: One or more processing systems, comprising: one or more memories comprising computer-executable instructions; and one or more processors configured to execute the computer-executable instructions and cause the one or more processing systems to perform a method in accordance with any one of clauses 1-24.

Clause 26: One or more processing systems, comprising means for performing a method in accordance with any one of clauses 1-24.

Clause 27: One or more non-transitory computer-readable media comprising instructions that, when executed by one or more processors of a computing system, cause the computing system to perform the operations of any one of clauses 1-24.

Clause 28: One or more computer program products embodied on one or more computer-readable storage media comprising code for performing a method in accordance with any one of clauses 1-24.

Clause 34. A vehicle comprising a vehicle body; one or more sensors; and a processing system coupled to the one or more sensors, the processing system comprising a processor configured to execute computer-executable instructions and cause the processing system to obtain physiological signal data of a person present in the vehicle body from the one or more sensors; determine an emotional state of the occupant based on phase segmented biomarker data of the physiological signal data; and enforce a response mechanism comprising one or more of an output of an audio-visual alert, an output of a haptic alert, change in vehicular control, or an adjustment of vehicle movement based on the physiological state.

Clause 35. The vehicle of clause 34, wherein the processor system further comprises a network interface coupled to a server system, and wherein the processor further causes the processing system to receive the phase segmented biomarker data.

Clause 36. The vehicle of any of clauses 34-35, wherein the processor further causes the processing system to: phase segment one or more biomarker data of the physiological signal data into general adaptation syndrome (GAS) phases, or send the physiological signal data to a server system to phase segment the one or more biomarker data into general adaptation syndrome (GAS) phases.

Clause 37. The vehicle of any of clauses 34-36, wherein the processor is configured to cause the processing system to: preprocess the physiological signal data to remove motion artifacts; extract, via signal processing, one or more biomarker data from the physiological signal data; phase segment a first biomarker data of the one or more biomarker data into one or more general adaptation syndrome (GAS) phases; and generate one or more respective similarity scores for each GAS phase of the one or more GAS phases based on at least one of a similarity to other GAS phases of the GAS phases or on a similarity to baseline data.

Clause 38. The vehicle any of clauses 34-37, wherein to phase segment, comprises the processor to further cause the processing system to: identify a first GAS phase of a first biomarker data of the one or more biomarker data; and identify a second GAS phase of the first biomarker data of the one or more biomarker data.

Clause 39. The vehicle any of clauses 34-38, wherein to enforce the response mechanism comprises the processor to further cause the processing system to output an audio visual alert, adjust lighting in the vehicle, adjust seat positions, adjust seat belts, or take control of the vehicle, adjusting a hospital bed, a drug dosage, or output of information to a hospital system.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, unless stated otherwise, the term “or” is used in an inclusive sense. This inclusive usage of or is equivalent to “and/or”. Thus, when options are delineated using “or” it permits the selection of one or more of the enumerated options concurrently. For example, if the document stipulates that a component may comprise option A or option B, it shall be understood to mean that the component may comprise option A, option B, or both option A and option B, and does not mean, unless stated expressly that the component includes either option A or option B. This inclusive interpretation ensures that all potential combinations of the options are permissible, rather than restricting the choice to a singular, exclusive option.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
obtaining physiological signal data;
preprocessing the physiological signal data to remove motion artifacts;
extracting, via signal processing, one or more biomarker data from the physiological signal data; and
phase segmenting a first biomarker data of the one or more biomarker data into general adaptation syndrome (GAS) phases.

2. The method of claim 1, wherein the phase segmenting comprises:
identifying a first GAS phase of the first biomarker data; and
marking the first GAS phase of the first biomarker data based on at least one of a start or an end of the first GAS phase.

3. The method of claim 2, wherein the first GAS phase is one of a first normal level phase, an alarm phase, a resistance phase, an exhaustion phase and a second normal level phase.

4. The method of claim 2, wherein the phase segmenting further comprises:
identifying a second GAS phase of the first biomarker data; and
marking the second GAS phase of the first biomarker data based on at least one of a start or an end of the second GAS phase.

5. The method of claim 1, further comprising:
generating one or more respective similarity scores for each GAS phase of the GAS phases based on at least one of a similarity to other GAS phases of the GAS phases or on a similarity to baseline data.

6. The method of claim 2, wherein the first GAS phase is a resistance phase, wherein identifying the first GAS phase as the resistance phase is based on a similarity score of the resistance phase being lower than a threshold.

7. The method of claim 1, wherein the phase segmentation comprises at least one of a parametric technique, a non-parametric technique, or a kernel-based technique applied to the one or more biomarker data, and wherein the phase segmenting is based on at least one of a duration or a distribution of the first biomarker data.

8. The method of claim 1, further comprising labeling the first biomarker data based on the GAS phase to generate first labeled data.

9. The method of claim 8, further comprising:
adding the first labeled data to a labeled data set; and
training a machine learning model on the labeled data set.

10. The method of claim 1, further comprising at least one of:
associating the GAS phases with one or more stressors, wherein the associating is based on a sensor reading or a user response to a push notification or other information request; or
temporally aligning various biomarkers of the first biomarker data based on onset of phases.

11. The method of claim 1, further comprising phase segmenting a second biomarker data of the one or more biomarker data into GAS phases.

12. The method of claim 1, wherein the physiological signal data is obtained from at least one of one or more sensors comprising one or more of cardiovascular sensors, respiratory sensors, neurological sensors, metabolic sensors, glucose sensors, or motion sensors.

13. The method of claim 1, further comprising alerting one or more users based on an identified phase of the first biomarker data, wherein the identified phase is associated with a health status or an emotional status or outputting an audio-visual alert of health status information to one or more devices based on an identified phase of the first biomarker data.

14. A processing system, comprising:
a memory comprising computer-executable instructions; and
a processor configured to execute the computer-executable instructions and cause the processing system to:
obtain physiological signal data;
preprocess the physiological signal data to remove motion artifacts;
extract, via signal processing, one or more biomarker data from the physiological signal data;
phase segment a first biomarker data of the one or more biomarker data into general adaptation syndrome (GAS) phases;
generate one or more respective similarity scores for each GAS phase of the one or more GAS phases based on at least one of a similarity to other GAS phases of the GAS phases or on a similarity to baseline data; and
label the first biomarker data based on the GAS phase to generate first labeled data.

15. A vehicle comprising:
a vehicle body;
one or more sensors; and
a processing system coupled to the one or more sensors, the processing system comprising a processor configured to execute computer-executable instructions and cause the processing system to:
obtain physiological signal data of an occupant of the vehicle from the one or more sensors;
determine an emotional state of the occupant based on phase segmented biomarker data of the physiological signal data; and
enforce a response mechanism comprising one or more of an output of an audio-visual alert, an output of a haptic alert, change in vehicular control, or an adjustment of vehicle movement based on the emotional state.

16. The vehicle of claim 15, wherein the processing system further comprises a network interface coupled to a server system, and wherein the processor further causes the processing system to receive the phase segmented biomarker data.

17. The vehicle of claim 15, wherein the processor further causes the processing system to:
phase segment one or more biomarker data of the physiological signal data to generate the phase segmented biomarker data comprising general adaptation syndrome (GAS) phases, or
send the physiological signal data to a server system to phase segment the one or more biomarker data to generate the phase segmented biomarker data comprising the GAS phases.

18. The vehicle of claim 16, wherein the processor is configured to cause the processing system to:
preprocess the physiological signal data to remove motion artifacts;
extract, via signal processing, one or more biomarker data from the physiological signal data;
phase segment a first biomarker data of the one or more biomarker data into one or more general adaptation syndrome (GAS) phases; and
generate one or more respective similarity scores for each GAS phase of the one or more GAS phases based on at least one of a similarity to other GAS phases of the GAS phases or on a similarity to baseline data.

19. The vehicle of claim 18, wherein to phase segment, the processor further causes the processing system to:
identify a first GAS phase of a first biomarker data of the one or more biomarker data; and
identify a second GAS phase of the first biomarker data of the one or more biomarker data.

20. The vehicle of claim 18, wherein to enforce the response mechanism the processor further causes the processing system to output an audio visual alert, adjust lighting in the vehicle, adjust seat positions, adjust seat belts, or take control of the vehicle.
